(19) United States
(12) Patent Application Publication
(10) Pub. No.: US 2021/0213370 A1
(43) Pub. Date: Jul. 15, 2021

(54) POWER SUPPLY MAGIC BOX

(71) Applicant: Zhenping Huading Stainless Steel Products Co. Ltd., Nanyang City (CN)

(72) Inventor: Hengzhong Wang, Shanghai City (CN)

(21) Appl. No.: 16/991,737

(22) Filed: Aug. 12, 2020

(30) Foreign Application Priority Data
Jan. 13, 2020 (CN) 202020077304.9

(51) Int. Cl. A63J 21/00; A63H 33/26

(52) U.S. Cl. CPC A63J 21/00; A63H 33/26

(57) ABSTRACT

A power supply magic box includes a box body and a box cover provided on the box body, wherein the box cover is provided on the top of the box body; the box body is provided with a first vertical plate and a second vertical plate, the vertical plate divides the box body into a first accommodating cavity and a second accommodating cavity, the second vertical plate is located inside the second accommodating cavity, and the second vertical plate divides the second accommodating cavity into a first accommodating part and a second accommodating part; the box body is provided with a controller and a circuit board, the circuit board is electrically connected to the controller; the box body is further provided with a voltage regulating knob, a voltage display screen and a touch switch, the voltage display screen and the touch switch are both electrically connected with the circuit board.

### CROSS REFERENCE TO RELATED APPLICATION

This application takes priority from and claims the benefit of Chinese Patent Application Ser. No. 202020077304.9 filed on Jan. 13, 2020, the contents of which are herein incorporated by reference.

### TECHNICAL FIELD

The present invention relates to the technical field of tattoo appliances, in particular to a power supply magic box.

### BACKGROUND OF THE INVENTION

In the process of tattooing, the tattoo artist needs to use parts such as a tattoo machine, a connecting line, a power supply, and a pedal.

The applicant found that the prior art has at least the following technical problems.

The existing tattoo tools are connected one by one during use due to too many parts and connecting lines, which is a waste of time, is not easy to store and carry, inconvenient to use, time-consuming and laborious to connect and use, and poor in practicability.

### SUMMARY OF THE INVENTION

The object of the present invention is to provide a power supply magic box, solving the technical problem in the prior art that the existing tattoo tools are connected one by one during use due to too many parts and connecting lines, which is a waste of time, is not easy to store and carry, inconvenient to use, time-consuming and laborious to connect and use, and poor in practicability. Many technical effects that can be produced by the preferred technical solutions among many technical solutions provided by the present invention are described in detail hereinafter.

In order to achieve the above object, the present invention provides the following technical solutions.

The present invention provides a power supply magic box, comprising a box body and a box cover provided on the box body, wherein:

the box cover is provided on the top of the box body;

the box body is provided with a first vertical plate and a second vertical plate, the vertical plate divides the box body into a first accommodating cavity and a second accommodating cavity, the second vertical plate is located inside the second accommodating cavity, and the second vertical plate divides the second accommodating cavity into a first accommodating part and a second accommodating part;

the box body is provided with a controller and a circuit board, the circuit board is electrically connected to the controller;

the box body is further provided with a voltage regulating knob, a voltage display screen and a touch switch, the voltage display screen and the touch switch are both electrically connected with the circuit board, and the pressure regulating knob is electrically connected to the controller.

Preferably, the controller is provided in the first accommodating cavity, and the circuit board is provided in the second accommodating part.

Preferably, the volume of the first accommodating part is greater than the volumes of the first accommodating cavity and the second accommodating part, and a tattoo tool is provided in a first accommodating part.

Preferably, the tattoo tool comprises a tattoo pen, and the tattoo pen is electrically connected to the circuit board.

Preferably, the bottom of the box cover is slidingly connected to the top of the box body.

Preferably, the power supply magic box further comprises a USB-C interface, wherein the USB-C interface is electrically connected to the controller.

Preferably, the box cover is further provided with a heat dissipation part, the heat dissipation part is provided to comprise a plurality of heat dissipation holes, the heat dissipation part is provided at the top of the USB-C interface, and the heat dissipation part is connected with the first accommodating cavity.

Preferably, the first accommodating cavity further comprises a power source, and the power source supplies power to the controller, the circuit board, the voltage regulating knob, and the voltage display screen and the touch switch.

Preferably, the power supply uses a storage battery.

Preferably, the box body is configured as a rectangle.

In the power supply magic box provided by the present invention, the inner cavity of the box body is divided into a first accommodating cavity and a second accommodating cavity by a first vertical plate, the second accommodating cavity is divided into a first accommodating part and a second accommodating part by a second vertical plate, the first accommodating cavity, the first accommodating part, and the second accommodating part are respectively provided with a controller, a power supply, a circuit board, a tattoo tool, etc., and a voltage regulating knob, a voltage display screen, and a touch switch are provided outside of the box body. This power supply magic box integrates the functions of the tattoo machine, the connection line, the power supply, and the pedal. Various parts do need to be connected one by one during use and can be used as normal directly. It is easy to carry and convenient to store. At the same time, the power supply magic box has a power supply function and a pedal function, which can directly replace the power supply and the foot switch.

### BRIEF DESCRIPTION OF THE DRAWINGS

In order to more clearly explain the embodiments of the present invention or the technical solutions in the prior art, the drawings required in the embodiments will be briefly introduced hereinafter. Obviously, the drawings in the following description are only a part of embodiments of the present invention. For those skilled in the art, other drawings may be obtained based on these drawings without paying any creative labor.

FIG. 1 is a schematic structural diagram of an embodiment of a power supply magic box according to the present invention;

FIG. 2 is a top schematic structural diagram of an embodiment of a power supply magic box according to the present invention;

FIG. 3 is a schematic cross-sectional diagram of A-A in FIG. 2;

FIG. 4 is a front schematic structural diagram of an embodiment of a power supply magic box according to the present invention;

FIG. 5 is a left schematic structural diagram of an embodiment of a power supply magic box according to the present invention.

In the figure: 1, box body; 2, first vertical plate; 3, second vertical plate; 4, controller; 5, voltage regulating knob; 6, circuit board; 7, voltage display screen; 8, touch switch; 9, box cover; 10, first accommodating cavity; 11, heat dissipation part; 12, USB-C interface; 20, second accommodating cavity; 111, heat dissipation hole; 201, first accommodating part; 202, second accommodating part.

### DETAILED DESCRIPTION OF THE SEVERAL EMBODIMENTS

In order to make the objectives, technical solutions and advantages of the present invention clearer, the technical solutions of the present invention will be described in detail below. Obviously, the described embodiments are only a part of embodiments of the present invention, rather than all the embodiments. Based on the embodiments of the present invention, all other embodiments obtained by those skilled in the art without paying any creative labor shall fall within the protection scope of the present invention.

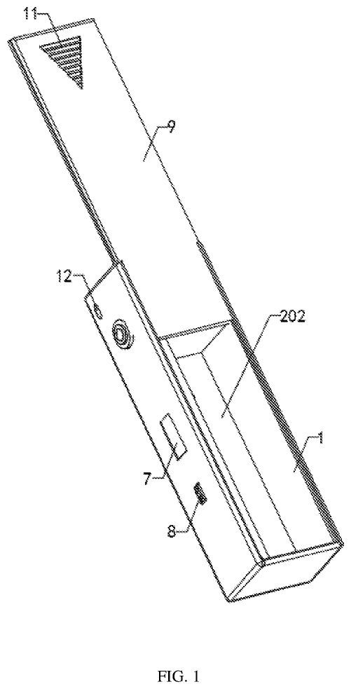

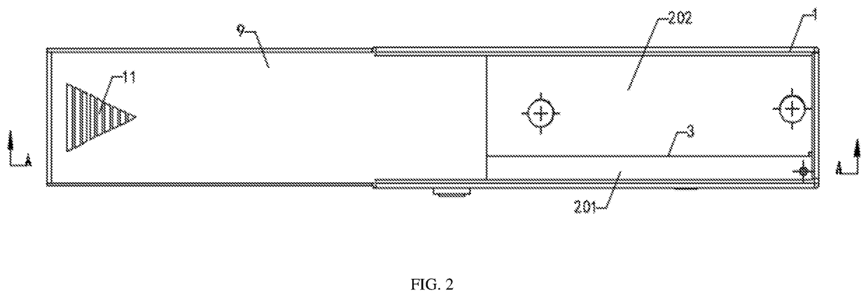

The present invention provides a power supply magic box. FIG. 1 is a schematic structural diagram of this embodiment; and FIG. 2 is a top schematic structural diagram of FIG. 1. As shown in FIGS. 1 and 2, the power supply magic box comprises a box body 1 and a box cover 9 provided on the box body 1, wherein

the box cover 9 is provided on the top of the box body 1, and the box body 1 is provided with an inner cavity.

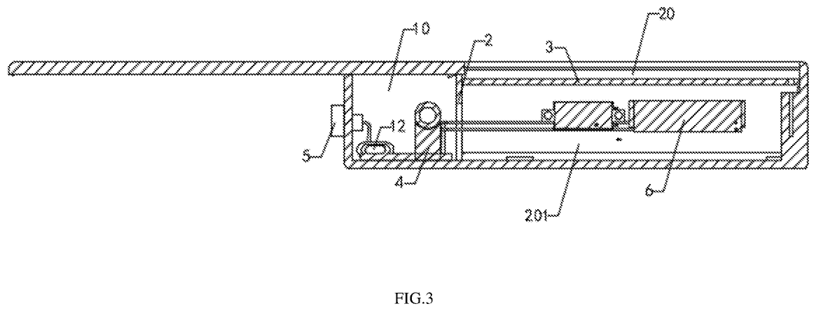

FIG. 3 is a schematic cross-sectional diagram of A-A in FIG. 2. As shown in FIG. 3, the box body 1 is provided with a first vertical plate 2 and a second vertical plate 3, the vertical plate 2 divides the box body 1 into a first accommodating cavity 10 and a second accommodating cavity 20, the second vertical plate 3 is located inside the second accommodating cavity 20, and the second vertical plate 3 divides the second accommodating cavity 20 into a first accommodating part 201 and a second accommodating part 202.

Specifically, the first vertical plate 2 is provided on the left part of the inner cavity, and divides the inner cavity of the box body 1 into a left cavity and a right cavity, namely, a first accommodating cavity 10 and a second accommodating cavity 20. The second vertical plate 3 is located in the second accommodating cavity 20 on the right side. The second accommodating cavity 20 is divided into a front cavity and a rear cavity, namely, a first accommodating part 201 and a second accommodating part 202.

The box body 1 is provided with a controller 4 and a circuit board 6, and the circuit board 6 is electrically connected to the controller 4. Specifically, the controller 4 is provided in the first accommodating cavity 10, and the circuit board 6 is provided in the second accommodating part 202. When in use, the controller 4 is connected to the power source.

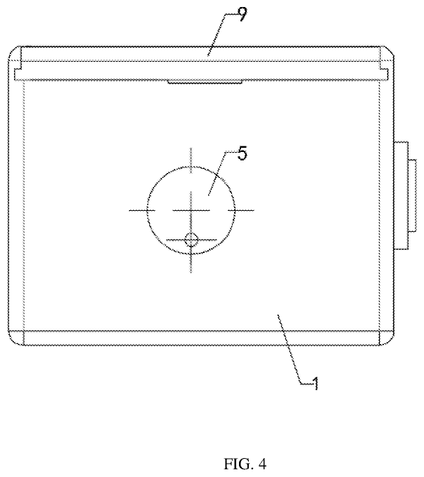

FIG. 4 is a left schematic structural diagram of FIG. 1. As shown in FIG. 4, the box body 1 is further provided with a voltage regulating knob 5, a voltage display screen 7 and a touch switch 8.

Specifically, the pressure regulating knob 5 is provided outside the box body, the right end of the pressure regulating knob 5 is connected with the left end of the box body 1, and the pressure regulating knob 5 is electrically connected with the controller 4.

Both the voltage display screen 7 and the touch switch 8 are electrically connected to the circuit board 6. The circuit board 6 is installed in the cavity on the front side of the second vertical plate 3, and the voltage display screen 7 and the touch switch 8 are both provided outside the box body 1, and specifically, are located at the front end of the box body 1, so as to be connected with the circuit board. The touch switch 8 is provided to replace the existing foot switch, which is convenient and practical. The pressure regulating knob 5 is electrically connected to the controller 4.

The power supply magic box integrates the functions of the tattoo machine, the connection line, the power supply, and the pedal. Various parts do need to be connected one by one during use and can be used as normal directly. It is easy to carry and convenient to store. At the same time, the power supply magic box has a power supply function and a pedal function, which can directly replace the power supply and the foot switch.

The working principle of this power supply magic box is as follows: when a tattoo is needed, the operator connects the power supply magic box to the power supply and then opens the box cover 9, takes out the needed related tools from the back cavity of the box body 1, that is, the first accommodating part 201, and then rotates the voltage regulating knob 5 to adjust the voltage according to the working content, so that the power supply magic box starts to work through the touch switch 8, and the operator can use the tattoo tool to work.

As an alternative embodiment, the volume of the first accommodating part 201 is greater than the volumes of the first accommodating cavity 10 and the second accommodating part 202, and a tattoo tool is provided in the first accommodating part 201. The tattoo tool may be a tattoo pen, an eyebrow tattoo pens or other tattoo tools, auxiliary tools, etc. The tattoo tool is provided in the first accommodating part 201, which is convenient to carry the tattoo tool with the box body 1 and is strong in storage ability, available at any time, is convenient and quick.

As an alternative embodiment, the tattoo tool comprises a tattoo pen, and the tattoo pen is electrically connected to the circuit board 6. A tattoo pen is placed in the power supply magic box and is connected to the box body 1. A circuit board 6 is placed in the power supply magic box to be plugged for operation. In the process of tattooing, the tattoo artist can directly take out and use the tattoo pen. There is no need to connect various connecting wires and other components one by one, which avoids a waste of time when using the existing tattoo tool. In this embodiment, the tattoo pen is directly placed in the first accommodating part 201, which is convenient to store and carry. When in use, the box body 1 can be opened by sliding the box cover 9 and is available at any time, high in practicability, easy and convenient to use.

As an alternative embodiment, the bottom of the box cover 9 is slidably connected with the top of the box body 1. A sliding cover is provided, the box cover 9 and the box body 1 are provided as a whole. The box cover 9 directly slides to open the box body 1 during use. After the contents of the box body 1 are taken out, the box cover 9 can be quickly closed by pushing and pulling, which has good airtightness and avoids the risk of the box cover 9 falling off. It is simple to use and easy to operate, which improves the working efficiency of the tattoo artist and the safety of the tattoo process.

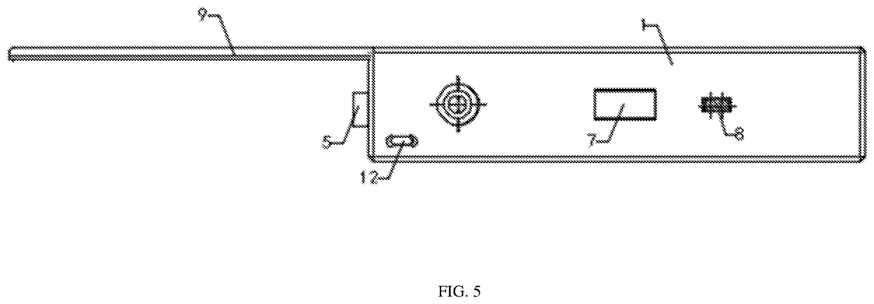

As an alternative embodiment, FIG. 5 is a schematic diagram of the main structure of this embodiment. As shown in FIG. 5, the power supply magic box further comprises a USB-C interface 12, and the USB-C interface 12 is electrically connected to the controller 4. The USB-C interface 12 is provided, the power supply magic box can be connected to external signals, enhancing the function of the power supply magic box, and improving the practicality.

As an alternative embodiment, the box cover 9 is further provided with a heat dissipation part 11, the heat dissipation part 11 is provided to comprise a plurality of heat dissipation holes 111, the heat dissipation part 11 is provided at the top of the USB-C interface 12, and the heat dissipation part 11 is provided to be connected with the first accommodating cavity 10. In this embodiment, the heat dissipation part 11 uses a heat dissipation mesh, and the heat dissipation mesh is provided to be connected with the first accommodating cavity 10, so as to avoid the problem that the long working time of this power supply magic box easily results in excess temperature of the first accommodating cavity 10 of the box body 1 so that electrical components burn out. By providing a heat dissipation part 11, the air circulation in the first accommodating cavity 10 can be enhanced, the temperature can be appropriately reduced, and the service life of the entire power supply magic box of the device and the working stability can be increased.

As an alternative embodiment, the first accommodating cavity 10 further comprises a power supply, and the power supply supplies power to the controller 4, the circuit board 6, the voltage regulating knob 5, the voltage display screen 7 and the touch switch 8. A power supply is provided in the first accommodating cavity 10 of the box body 1. Specifically, a storage battery is used for the power supply, which can be used after charging the power supply magic box in advance, or can be used while charging, so as to meet the requirements of different use occasions. When in use, the tattoo machine, the connecting line, the power supply, and the foot switch required for the tattoo are easily replaced by a power supply magic box. It is easy to use, portable and high in practicability. Of course, this tattoo can also be plugged to provide electric energy.

As an alternative embodiment, the box body 1 is configured as a rectangle. In actual use, the shape of the box body 1 can also be configured into a regular shape such as a square, a polygon, a rounded rectangle, etc., or can be designed or customized according to the actual needs of the tattoo artist. In this embodiment, the box body 1 of the power supply magic box is configured as a rectangle, which can facilitate the placement of tools such as tattoo pens and eyebrow tattoo pens, and improve the practicability.

In the power supply magic box of the present invention, the installation method, the connection method or the configuring method are all common mechanical methods, and it can be implemented as long as the beneficial effects can be achieved.

The above are only specific embodiments of the present invention, but the protection scope of the present invention is not limited thereto. Any changes or substitutions conceivable to those skilled in the art within the technical scope disclosed by the present invention should be covered within the protection scope of the present invention. Therefore, the protection scope of the present invention should be subject to the protection scope of the claims.

What is claimed is:

1. A power supply magic box, comprising a box body and a box cover provided on the box body, wherein
the box cover is provided on the top of the box body;
the box body is provided with a first vertical plate and a second vertical plate, the vertical plate divides the box body into a first accommodating cavity and a second accommodating cavity, the second vertical plate is located inside the second accommodating cavity, and the second vertical plate divides the second accommodating cavity into a first accommodating part and a second accommodating part;
the box body is provided with a controller and a circuit board, the circuit board is electrically connected to the controller;
the box body is further provided with a voltage regulating knob, a voltage display screen and a touch switch, the voltage display screen and the touch switch are both electrically connected with the circuit board, and the pressure regulating knob is electrically connected to the controller.

2. The power supply magic box according to claim 1, wherein the controller is provided in the first accommodating cavity, and the circuit board is provided in the second accommodating part.

3. The power supply magic box according to claim 1, wherein the volume of the first accommodating part is greater than the volumes of the first accommodating cavity and the second accommodating part, and a tattoo tool is provided in a first accommodating part.

4. The power supply magic box according to claim 3, wherein the tattoo tool comprises a tattoo pen, and the tattoo pen is electrically connected to the circuit board.

5. The power supply magic box according to claim 1, wherein the bottom of the box cover is slidingly connected to the top of the box body.

6. The power supply magic box according to claim 1, further comprising a USB-C interface, wherein the USB-C interface is electrically connected to the controller.

7. The power supply magic box according to claim 6, wherein: the box cover is further provided with a heat dissipation part, the heat dissipation part is provided to comprise a plurality of heat dissipation holes, the heat dissipation part is provided at the top of the USB-C interface, and the heat dissipation part is connected with the first accommodating cavity.

8. The power supply magic box according to claim 1, wherein the first accommodating cavity further comprises a power source, and the power source supplies power to the controller, the circuit board, the voltage regulating knob, and the voltage display screen and the touch switch.

9. The power supply magic box according to claim 8, wherein the power supply uses a storage battery.

10. The power supply magic box according to claim 1, wherein the box body is configured as a rectangle.
